(19) United States
(12) Patent Application Publication
(10) Pub. No.: US 2011/0092930 A1
(43) Pub. Date: Apr. 21, 2011

(54) MEDICAL PROTECTIVE TABLE SHEETS

(75) Inventor: Christopher D. Poorman, St. Helens, OR (US)

(73) Assignee: TriMountain Medical Resources LLC, St. Helens, OR (US)

(21) Appl. No.: 12/582,021

(22) Filed: Oct. 20, 2009

(51) Int. Cl. A61F 13/15; A47G 9/00

(52) U.S. Cl. 604/356; 5/484

(57) ABSTRACT

A disposable slip-resistant and preferably absorbent protective medical table sheet for protecting a medical table, such as an operating table or an examination table, from contamination due to contact with bodily fluids or other contaminants associated with a patient positioned upon the table sheet covering the medical table, and for resisting slippage between the patient and the table sheet and between the table sheet and the medical table and, if a lift sheet is used, between the table sheet and the lift sheet. The table sheet preferably includes an impermeable layer with a slip-resistant and absorbent layer of foam material on its upper side and a distribution of slip-resistant material on its lower side. The lower side of the impermeable layer may itself be comprised of slip-resistant material. The table sheet is preferably fan-and-book-foldable into a compact yet rapidly unfoldable table sheet.

### CROSS-REFERENCE TO RELATED APPLICATIONS

None.

### BACKGROUND OF THE INVENTION

This disclosure relates generally to table sheets adapted for use in hospital or medical applications, and, more particularly, to disposable table sheets designed for use as protective table sheets in hospital operating room or patient examination environments where non-slip, impermeability, and absorbency capabilities are desirable.

A hospital operating room or patient examination table typically includes a relatively thin mattress upon which a patient may be positioned for examination or medical attention. A reusable woven fabric sheet or “linen” may be used to cover and protect the table mattress. Such linens are commonly used and provide limited absorption of bodily fluids from the patient. However, linens must be laundered for successive use and can allow fluids to strike through to the mattress below, increasing the potential for cross-contamination from one patient to the next.

Alternatively, a disposable protective table sheet may be used to help protect the table and mattress from becoming soiled during the examination or medical procedure, to allow for rapid cleanup and preparation of the table for a subsequent patient, and to minimize the potential for cross-contamination from one patient to the next. Currently available disposable protective table sheets are typically made of plastic (non-absorbent) material on a bottom side (for contact with the table mattress) and a very thin (very low absorbent) non-woven paper or fabric layer on the top side.

The protective table sheet is placed to cover the mattress of the operating or examination table. A semi-absorbent lift (or draw) sheet is typically placed cross-wise over the table sheet so as to cover an area of the table immediately under the patient's torso. During the medical procedure, the patient lies on the table sheet with the lift sheet under the patient's mid-section or torso area. Outward edges of the lift sheet may be gripped by the medical practitioners for lifting and repositioning the patient during the examination or medical procedure.

During the examination or medical procedure, the patient may be inadvertently repositioned by the patient's own movements or in reaction to activities of the medical practitioners or, for example, by gravity if the table is inclined. In typical hospital operating room or patient examination environments, currently available disposable protective table sheets allow slippage between the top surface of the table mattress pad and the bottom surface of the disposable table sheet and/or between the top surface of the disposable table sheet and the bottom surface of the lift sheet and/or between the top surface of the disposable table sheet and the skin of the patient. The inadvertent slippage may lead to difficulty keeping the patient positioned correctly during a medical procedure. Thus, improved disposable protective table sheets are needed for operating and examination table applications where non-slip, impermeability, and absorbency capabilities are desirable.

### BRIEF DESCRIPTION OF THE SEVERAL DRAWINGS

For a more complete understanding of the present invention, the drawings herein illustrate examples of the invention. The drawings, however, do not limit the scope of the invention. Similar references in the drawings indicate similar elements.

FIG. 1 is a sectional side view of an exemplary sheet in accordance with one embodiment of the present invention.

FIG. 2 is a sectional side view of an exemplary sheet in accordance with an alternate embodiment of the present invention.

FIG. 3A is a perspective view of an exemplary sheet in accordance with various embodiments.

FIG. 3B is a perspective view of the sheet in FIG. 3A shown partially folded.

FIG. 3C is a perspective view of the partially folded sheet in FIG. 3B shown further partially folded.

FIG. 3D is a perspective view of the partially folded sheet in FIG. 3C shown further folded.

### DETAILED DESCRIPTION OF PREFERRED EMBODIMENTS

In the following detailed description, numerous specific details are set forth in order to provide a thorough understanding of various embodiments. However, those skilled in the art will understand that the present invention may be practiced without these specific details, that the present invention is not limited to the described embodiments, and that the present invention may be practiced in a variety of alternate embodiments. In other instances, well known methods, procedures, components, and systems have not been described in detail.

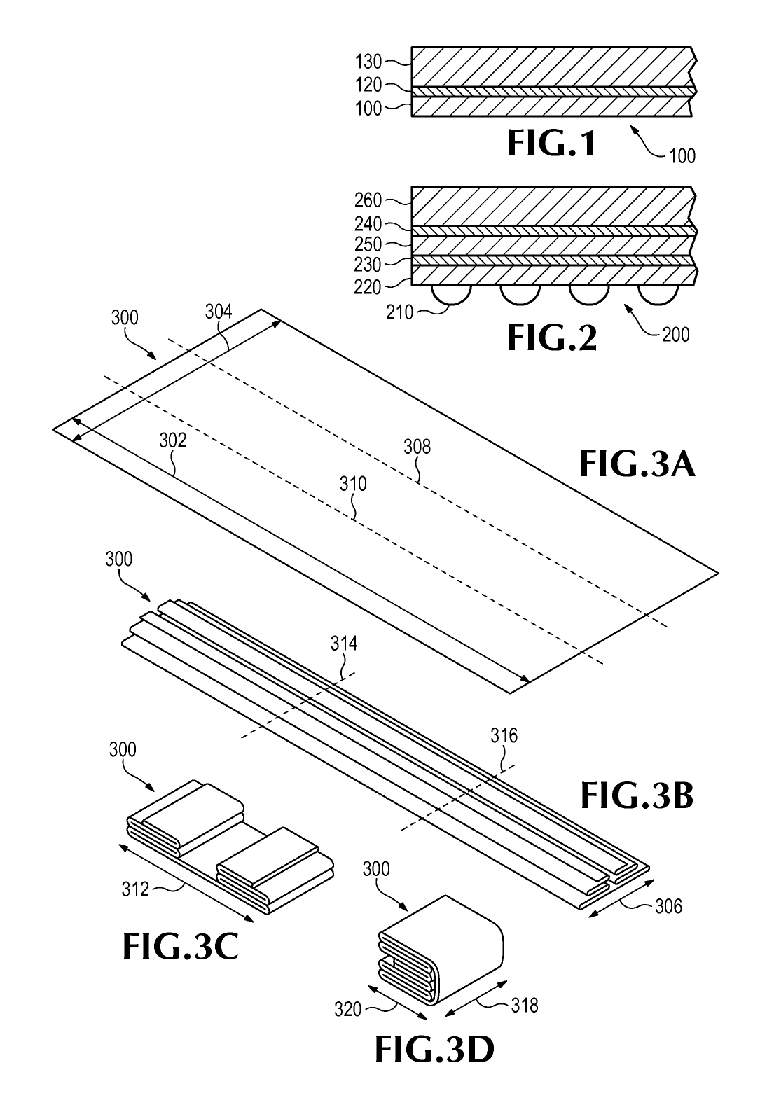

As shown in the sectional side view in FIG. 1, an exemplary table sheet 100 in accordance with one embodiment of the present invention may comprise a disposable slip-resistant and absorbent protective medical table sheet 100 for protecting a medical table (not shown), such as, for example, an operating table or an examination table, from contamination due to contact with bodily fluids or other contaminants associated with a patient that is positioned upon (i.e. on top of) the table sheet 100 that is in turn covering the medical table therebelow. The present inventor discovered that existing disposable table sheets fail to resist slippage between a patient positioned upon the table sheet and the top surface of the table sheet therebelow and, if a lift sheet is used, slippage between the top surface of the table sheet and the bottom surface of the lift sheet, and that existing table sheets fail to resist slippage between the lower surface or underside of the table sheet and the top surface of a medical table covered by the table sheet. The present inventor further discovered that existing disposable table sheets typically consist of a plastic sheet having a very thin non-woven paper scrim top surface that neither provides slip-resistance for a patient positioned upon the table sheet (and/or upon a lift sheet positioned upon the table sheet) nor effective absorbency of bodily fluids or other (typically fluid) contaminants associated with the patient. Instead, such existing table sheets provide slippery upper surfaces that easily slip beneath a patient's skin, the bottom surface of a lift sheet, if used, and any outer garments, and such existing table sheets provide slippery lower surfaces that easily slip on the top surface of the medical table. The top surface of a medical table is typically a vinyl or similar material covering of the table mattress but may also consist of other textile materials or even a mattress-less or pad-less hard surface such as wood or metal.

The present inventor discovered and invented a disposable table sheet having an impermeable layer 110 that has an upper side (directed upward toward, as shown in FIG. 1, optional adhesive coating 120 and slip-resistant and absorbent material 130) and a lower side (directed downward to a medical table top surface (not shown)) and having length 302 and width 304 dimensions (as for the table sheet 300 in FIG. 3A) sufficient to substantially cover at least the top surface of the medical table. The impermeable layer 110, which may comprise a composite of component layers, is preferably capable of preventing the passage of fluids (such as bodily fluids or other contaminants associated with a patient positioned upon the table sheet 100) from the upper side of the impermeable layer 110 to the lower side of said impermeable layer 110. In one embodiment, the impermeable layer 110 is made of a foam urethane.

The slip-resistant and absorbent material 130 preferably comprises upwardly oriented slip-resistant and absorbent material (such as a PU (polyurethane) foam material) fixedly secured (optionally using an adhesive coating 120) to at least a portion of the upper side of the impermeable layer 110. Preferably the portion treated with the slip-resistant and absorbent material 130 covers at least the portion of the impermeable layer 110 upon which a patient is positioned for the intended medical procedure or examination. And most preferably, the slip-resistant and absorbent material 130 covers the entire upper side of the impermeable layer 110. That is, the slip-resistant material 130 fixedly secured to the upper side of the impermeable layer 110 may be in a distribution covering an area ranging from a small area of the impermeable layer 110 to substantially an entire area of said impermeable layer, or the slip-resistant material 130 fixedly secured to the upper side of the impermeable layer 110 may be in a contiguous layer covering an area ranging from the small area of said impermeable layer to substantially the entire area of the impermeable layer 110. Various methods of fixedly securing the slip-resistant and absorbent material 130 may be used. For example, sonic or heat welding may be used to fixedly secure the slip-resistant and absorbent material 130 to the impermeable layer 110.

Preferably, the upwardly oriented slip-resistant and absorbent material 130 is capable of resisting slippage between the patient and the table sheet and also between a lift sheet and the table sheet when the patient is positioned upon the lift sheet and when the lift sheet is positioned upon the portion of the upper side of the impermeable layer 110 having the upwardly oriented slip-resistant and absorbent material 130 attached thereto. As previously described, a lift (or draw) sheet is commonly used in combination with a protective table sheet to facilitate repositioning the patient. Such a lift sheet is commonly a rectangular sheet made of rayon-polyester or similar blend of material having sufficient tensile strength to allow for lifting up and repositioning the patient during a medical procedure or examination. The present inventor discovered that the top surface of existing table sheets fails to provide slip-resistance between the table sheet and the lift sheet and that, as in a preferred embodiment, polyurethane foam used for the slip-resistant and absorbent material 130 desirably resists slippage between the table sheet and typical lift sheets and is also capable of absorbing at least some of the bodily fluids or other contaminants associated with a patient positioned upon the table sheet 100.

The present inventor further discovered that, as in one embodiment, foam urethane used for the impermeable layer 110 desirably prevents strike through of bodily fluids or other fluid contaminants from the upper side to the lower side of the impermeable layer 110 and also resists slippage between the table sheet 100 and the top surface of the medical table when a patient is positioned upon the table sheet opposite said portion of said lower side of said impermeable layer. The present inventor yet further discovered that, as in one embodiment, a foam construction (such as PU (polyurethane) foam) for the upwardly oriented slip-resistant material desirably resists slippage when in contact with human skin and is capable of absorbing at least some of the bodily fluids or other contaminants associated with the patient. The upwardly oriented material 130 is preferably not irritating to human skin and also resists slippage with human skin.

In one embodiment, the table sheet preferably has sufficient tensile strength, whether by selection of the material or combination of components materials used to fabricate the impermeable layer 110 or to fabricate the upwardly oriented slip-resistant material 130, so that said patient is repositionable upon the medical table by lifting and repositioning the patient using the table sheet without use of a separate lift sheet.

An alternate embodiment is shown in the sectional side view of FIG. 2. As shown, the disposable table sheet 200 preferably comprises upwardly oriented slip-resistant material 260 fixedly secured (such as by an adhesive 240) to at least a portion of the upper side of an impermeable layer 250, 220 (which may comprise principally of a waterproof layer of PE (polyethylene) film 250 but which may also comprise of, as shown in FIG. 2, layers 250 and 220 with an adhesive coating 230 therebetween). The upwardly oriented slip-resistant material 260 is preferably capable of resisting slippage between a patient and the table sheet when the patient is positioned upon the portion of the upper side of the impermeable layer having the upwardly oriented slip-resistant material 260 attached thereto.

The impermeable layer 250, 220 is shown, in a preferred embodiment, to include a film layer 250 fixedly secured (such as by coating or adhesive 230) to a substrate layer 220, with the film layer 250 being on the upper side (of this impermeable layer) and the substrate 220 being on the lower side. This is principally to accommodate the downwardly oriented slip-resistant material 210 fixedly secured to at least a portion of the lower side of the impermeable layer 250, 220. The downwardly oriented material 210, as shown in FIG. 2, comprise depositions of slip-resistant material (such as PVC material) on a substrate layer 220 (which may comprise a light-blue colored spunbond PET (polyester). However, the impermeable layer 250, 220 may comprise additional component layers or fewer component layers or a construction having, for instance, the film 250 below the substrate 220 instead of above the substrate 220 as shown in FIG. 2. For example, the substrate 220 and waterproof film 250 may share a single layer structure to which the downwardly oriented slip-resistant material 210 may be fixedly secured by such means as sonic or heat welding, pressure bonding, use of an adhesive, or other means, and to which the upwardly oriented slip-resistant material 260 may be fixedly secured.

The downwardly oriented material 210 is preferably capable of resisting slippage between the table sheet 200 and the top surface of a medical table when the patient is positioned upon the table sheet 200 opposite the portion of the lower side of the impermeable layer 250, 220 having the downwardly oriented slip-resistant material 210 attached thereto. The upwardly oriented slip-resistant material 260 is preferably capable of absorbing at least some of the bodily fluids or other contaminants associated with the patient. The upwardly oriented slip-resistant material 260 preferably comprises a foam construction that resists slippage when in contact with human skin, is capable of absorbing at least some of the bodily fluids or other contaminants associated with the patient, and is not irritating to human skin.

The downwardly oriented slip-resistant material 210 is shown in FIG. 2 as dots applied to the underside of the table sheet 200. The distribution of these depositions of slip-resistant material may be as shown, roughly covering 50% of the surface area of the lower side of the table sheet 200. However, the distribution of these depositions may range from a small area (i.e. a smaller area than what may be characterized as a 50% distribution shown in the sectional view in FIG. 2) to full coverage of the entire surface area of the lower side of the table sheet 200. Although not shown, the upwardly oriented slip-resistant material 260, which is shown in FIG. 2 in what may be characterized as a contiguous layer of material, may be distributed on the upper side as smaller dot-like depositions of slip-resistant material as shown for the downwardly oriented slip-resistant material 210. Likewise, the downwardly oriented slip-resistant material 210 may be distributed as a contiguous layer of material as shown for the upwardly oriented slip-resistant material 260. Similarly alternative distributions of the upwardly and downwardly oriented slip-resistant material shown in FIG. 1 may be used.

Preferably, the upwardly and downwardly oriented slip-resistant materials are fixedly secured to the upper and lower surfaces of the table sheet in at least those portions of the table sheet 300 shown in FIG. 3A in locations where a patient would be positioned for the intended medical procedure or examination. Preferably, the slip-resistant materials are fixedly secured in distributions that cover the entire upper and lower surface areas (i.e. each side of table sheet 300 having a surface area equal to the length 302 times the width 304) of the table sheet 300 (so as to maximize the slip-resistant benefits of the table sheet 300 in as many different applications as possible and to minimize manufacturing costs). In one embodiment, the distribution slip-resistant material may be concentrated to the region of the table sheet 300 between fold lines 308 and 310.

In one embodiment, the table sheet 300 preferably has sufficient tensile strength so that a patient is repositionable upon a medical table by lifting and repositioning the patient using the table sheet 300 without use of a separate lift sheet.

The disposable table sheet 300, shown in a flat orientation in FIG. 3A having a length 302 and a width 304, is preferably fan-foldable widthwise (i.e. along, for example, fold lines 308 and 310) into a widthwise fan-folded table sheet (as shown in FIG. 3B) of length 302 and a widthwise reduced width 306. The widthwise fan-folded table sheet (shown in FIG. 3B) of length 302 and widthwise reduced width 306 is preferably fan-foldable lengthwise (i.e. along, for example, fold lines 314 and 316) into a widthwise-then-lengthwise fan-folded table sheet (as shown in FIG. 3C) of width 306 and lengthwise reduced length 312. The widthwise-then-lengthwise fan-folded table sheet (shown in FIG. 3C) is preferably book-foldable lengthwise into a compact yet rapidly unfoldable table sheet (as shown in FIG. 3D) of width 318 (which is the same as width 306 in FIGS. 3B and 3C) and further lengthwise reduced length 320. The compact yet rapidly unfoldable table sheet shown in FIG. 3D is preferable rapidly unfoldable by opening the book-folded sheet, extending the sheet to its full length 302, and extending the sheet to its full width 306. In a less preferred embodiment (not shown), the table sheet 300 may be fan-folded by first fan-folding lengthwise, then widthwise, and then book-folded widthwise.

Exemplary approximate dimensions for a table sheet 300 include a length 302 of 90 inches, a width 304 of 36 inches, a widthwise fan-folded width 306 of 9.5 inches, a widthwise-then-lengthwise fan-folded length 312 of 20 inches, and a book-folded length 320 of 9.5 inches. The upwardly oriented slip-resistant and absorbent material 260 (or 130) may comprise polyurethane foam that is approximately 2.0 mm thick, extending upwardly away from the layers therebelow. The downwardly oriented slip-resistant material 210 may comprise PVC-based anti-slip dots having a diameter of approximately 1.8 mm and a distribution of approximately 5.5 mm from each dot center to the next dot center.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

What is claimed is:

1. A disposable slip-resistant protective medical table sheet for protecting a medical table, such as an operating table or an examination table, from contamination due to contact with bodily fluids or other contaminants associated with a patient positioned upon said table sheet covering said medical table, and for resisting slippage between said patient and said table sheet and between said table sheet and said medical table, said table sheet comprising:
(a) an impermeable layer having an upper side and a lower side and having length and width dimensions sufficient to substantially cover at least a top surface of said medical table, said impermeable layer capable of preventing the passage of fluids from said upper side to said lower side of said impermeable layer;
(b) upwardly oriented slip-resistant material fixedly secured to at least a portion of said upper side of said impermeable layer, said upwardly oriented slip-resistant material capable of resisting slippage between said patient and said table sheet when said patient is positioned upon said portion of said upper side having said upwardly oriented slip-resistant material attached thereto; and
(c) downwardly oriented slip-resistant material fixedly secured to at least a portion of said lower side of said impermeable layer, said downwardly oriented material capable of resisting slippage between said table sheet and said medical table when said patient is positioned upon said table sheet opposite said portion of said lower side of said impermeable layer having said downwardly oriented slip-resistant material attached thereto.

2. The table sheet of claim 1 wherein said upwardly oriented slip-resistant material is capable of absorbing at least some of said bodily fluids or other contaminants associated with said patient.

3. The table sheet of claim 2 wherein said upwardly oriented slip-resistant material comprises a foam construction that resists slippage when in contact with human skin and is capable of absorbing at least some of said bodily fluids or other contaminants associated with said patient.

4. The table sheet of claim 1 wherein said upwardly oriented material is not irritating to human skin.

5. The table sheet of claim 4 wherein said upwardly oriented material resists slippage with human skin.

6. The table sheet of claim 1 wherein said upwardly oriented material resists slippage between a lift sheet and said table sheet when said lift sheet is placed between said table sheet and said patient.

7. The table sheet of claim 1 wherein said portion in (b) and (c) each, independently from one another, comprises: slip-resistant material fixedly secured to one or the other side of said impermeable layer in a distribution covering an area ranging from a small area of said impermeable layer to substantially an entire area of said impermeable layer; or slip-resistant material fixedly secured to one or the other side of said impermeable layer in a contiguous layer covering an area ranging from said small area of said impermeable layer to substantially said entire area of said impermeable layer.

8. The table sheet of claim 1 wherein said table sheet has sufficient tensile strength so that said patient is repositionable upon said medical table by lifting and repositioning said patient using said table sheet without use of a separate lift sheet.

9. The table sheet of claim 1 wherein a flat orientation of said table sheet of said length and said width is fan-foldable widthwise into a widthwise fan-folded table sheet of said length, said widthwise fan-folded table sheet of said length is fan-foldable lengthwise into a widthwise-then-lengthwise fan-folded table sheet, and said widthwise-then-lengthwise fan-folded table sheet is book-foldable into a compact yet rapidly unfoldable table sheet.

10. A disposable slip-resistant and absorbent protective medical table sheet for protecting a medical table, such as an operating table or an examination table, from contamination due to contact with bodily fluids or other contaminants associated with a patient positioned upon said table sheet covering said medical table, for resisting slippage between said patient and said table sheet and between said table sheet and said medical table, and for absorbing at least some of said bodily fluids or other contaminants associated with said patient, said table sheet comprising:
(a) an impermeable layer having an upper side and a lower side and having length and width dimensions sufficient to substantially cover at least a top surface of said medical table, said impermeable layer capable of preventing the passage of fluids from said upper side to said lower side of said impermeable layer;
(b) upwardly oriented slip-resistant and absorbent material fixedly secured to at least a portion of said upper side of said impermeable layer, said upwardly oriented slip-resistant and absorbent material capable of resisting slippage between said patient and said table sheet and between a lift sheet and said table sheet when said patient is positioned upon said lift sheet and when said lift sheet is positioned upon said portion of said upper side having said upwardly oriented slip-resistant and absorbent material attached thereto, said upwardly oriented slip-resistant and absorbent material capable of absorbing at least some of said bodily fluids or other contaminants associated with said patient; and
(c) downwardly oriented slip-resistant material fixedly secured to at least a portion of said lower side of said impermeable layer, said downwardly oriented material capable of resisting slippage between said table sheet and said medical table when said patient is positioned upon said table sheet opposite said portion of said lower side of said impermeable layer having said downwardly oriented slip-resistant material attached thereto.

11. The table sheet of claim 10 wherein said upwardly oriented slip-resistant material comprises a foam construction that resists slippage when in contact with human skin and is capable of absorbing at least some of said bodily fluids or other contaminants associated with said patient.

12. The table sheet of claim 10 wherein said upwardly oriented material is not irritating to human skin.

13. The table sheet of claim 12 wherein said upwardly oriented material resists slippage with human skin.

14. The table sheet of claim 10 wherein said portion in (b) and (c) each, independently from one another, comprises: slip-resistant material fixedly secured to one or the other side of said impermeable layer in a distribution covering an area ranging from a small area of said impermeable layer to substantially an entire area of said impermeable layer; or slip-resistant material fixedly secured to one or the other side of said impermeable layer in a contiguous layer covering an area ranging from said small area of said impermeable layer to substantially said entire area of said impermeable layer.

15. A disposable slip-resistant and absorbent protective medical table sheet for protecting a medical table, such as an operating table or an examination table, from contamination due to contact with bodily fluids or other contaminants associated with a patient positioned upon said table sheet covering said medical table, for resisting slippage between said patient and said table sheet and between said table sheet and said medical table, and for absorbing at least some of said bodily fluids or other contaminants associated with said patient, said table sheet comprising:
(a) an impermeable layer having an upper side and a lower side and having length and width dimensions sufficient to substantially cover at least a top surface of said medical table, said impermeable layer capable of preventing the passage of fluids from said upper side to said lower side of said impermeable layer;
(b) upwardly oriented slip-resistant and absorbent material fixedly secured to at least a portion of said upper side of said impermeable layer, said upwardly oriented slip-resistant and absorbent material capable of resisting slippage between said patient and said table sheet and between a lift sheet and said table sheet when said patient is positioned upon said lift sheet and when said lift sheet is positioned upon said portion of said upper side having said upwardly oriented slip-resistant and absorbent material attached thereto, said upwardly oriented slip-resistant and absorbent material capable of absorbing at least some of said bodily fluids or other contaminants associated with said patient; and
(c) said lower side of said impermeable layer being capable of resisting slippage between said table sheet and said medical table when said patient is positioned upon said table sheet opposite said portion of said lower side of said impermeable layer.

16. The table sheet of claim 15 wherein said upwardly oriented slip-resistant material comprises a foam construction that resists slippage when in contact with human skin and is capable of absorbing at least some of said bodily fluids or other contaminants associated with said patient.

17. The table sheet of claim 15 wherein said upwardly oriented material is not irritating to human skin.

18. The table sheet of claim 17 wherein said upwardly oriented material resists slippage with human skin.

19. The table sheet of claim 15 wherein said portion in (b) comprises: slip-resistant material fixedly secured to said upper side of said impermeable layer in a distribution covering an area ranging from a small area of said impermeable layer to substantially an entire area of said impermeable layer; or slip-resistant material fixedly secured to said upper side of said impermeable layer in a contiguous layer covering an area ranging from said small area of said impermeable layer to substantially said entire area of said impermeable layer.

20. The table sheet of claim 15 wherein said table sheet has sufficient tensile strength so that said patient is repositionable upon said medical table by lifting and repositioning said patient using said table sheet without use of a separate lift sheet.
